(19) United States
(12) Patent Application Publication
(10) Pub. No.: US 2011/0111233 A1
(43) Pub. Date: May 12, 2011

(54) INORGANIC NANOPARTICLE LABELING AGENT

(76) Inventors: Kazuya Tsukada, Kanagawa (JP); Takuji Aimiya, Tokyo (JP)

(21) Appl. No.: 13/002,491

(22) PCT Filed: Feb. 26, 2009
(86) PCT No.: PCT/JP2009/053534

§ 371 Date: Jan. 3, 2011

(30) Foreign Application Priority Data
Jul. 7, 2008 (JP) 2008-176748

(51) Int. Cl. C09K 11/00; B05D 5/00; B82Y 5/00; B82Y 15/00; B82Y 40/00

(52) U.S. Cl. 428/404; 427/215; 428/403; 977/773; 977/774; 977/927

(57) ABSTRACT

An inorganic nanoparticle labeling agent having adaptability for being employed as a labeled material in the field of biology and medical science and capable of emitting fluorescence at a stable emission intensity is disclosed, comprising inorganic nanoparticles which were surface-modified with an organic compound, wherein the inorganic nanoparticles exhibit an average particle size of 1 to 10 nm, the organic compound is a compound containing a polyethylene glycol chain, the average particle size D of the inorganic nanoparticle labeling agent is from 8 to 25 nm; and an amount M (mol) of the organic compound per inorganic nanoparticle and a length L (nm) of the organic compound measured from an inorganic nanoparticle surface meet the relationship represented by the following formula (I):

(M×10²²)×L/D=1.0−4.5 Formula (I)

### TECHNICAL FIELD

The present invention relates to inorganic nanoparticles, and in particular to an inorganic nanoparticle labeling agent in which the surfaces of inorganic semiconductor nanoparticles have been modified with an organic compound.

### TECHNICAL BACKGROUND

Recent advances in nanotechnology suggest the possibility of employing inorganic nanoparticles for detection, diagnosis, sensitiveness or other applications. Inorganic nanoparticles capable of interacting with a biological system have broadly attracted attention in the field of biology or medical science. Such inorganic nanoparticles are expected to be useful as a novel intravascular probe for both of sensitiveness (for example, imaging) or therapeutic purpose (for example, drug delivery).

A substance which is composed of a nanometer-sized semiconductor material and exhibits a quantum confinement effect, for example, a semiconductor nanoparticle, is generally called a quantum dot. Such a quantum dot, which is a small agglomerate of some ten nms and composed of some hundreds to some thousands of semiconductor atoms, emits an energy equivalent to the energy band gap of the quantum dot when absorbing light from an exciting source and reaching an energy-excited state.

Therefore, it is considered that controlling the size or material composition of a quantum dot can adjust the energy band gap, enabling to employ an energy of a wavelength band at various levels. Accordingly, in the field of biology or medical science, there has been expected development of technologies of applying such semiconductor nanoparticles as a labeling material to obtain various data regarding a chemical substance, a molecule or the like constituting a living cell.

In cases when employing inorganic nanoparticles or semiconductor nanoparticles as a labeling material in the field of biology or medical science, the inorganic nanoparticles have poor affinity and dispersibility within living tissue/cell and easily aggregate, producing problems such as accumulation in a living body. Further, there are also produced problems such that its inherent labeling function is lost when adsorbed to a targeted molecule. Accordingly, its intact structure cannot become a labeling material, so that there was studied surface modification to achieve affinity or bonding properties to biomolecules to solve the foregoing problems; however, it is in such a situation that high-level adaptability required of a labeling material or the like to know the dynamic state of a molecule is still insufficient (as described, for example, Patent documents 1 and 2).
Patent document 1: JP2006-517985W
Patent document 2: JP2007-178239A

### DISCLOSURE OF THE INVENTION

### Problem to be Solved in the Invention

The present invention has come into being in view of the foregoing problems and circumstances. One problem to be solved is to provide an inorganic nanoparticle labeling agent having adaptability for being employed as a labeled material in the field of biology and medical science and capable of emitting fluorescence at a stable emission intensity.

### Means for Solving the Problem

The foregoing problems related to the present invention was solved by the following constitutions:

1. An inorganic nanoparticle labeling agent comprising inorganic nanoparticles which were surface-modified with an organic compound, wherein the inorganic nanoparticles exhibit an average particle size of 1 to 10 nm, the organic compound is a compound containing a polyethylene glycol chain, the average particle size D of the inorganic nanoparticle labeling agent is from 8 to 25 nm; and an amount M (mol) of the organic compound per single inorganic nanoparticle and a length L (nm) of the organic compound, measured from the inorganic nanoparticle surface meet the relationship represented by the following formula (I):

(M×10²²)×L/D=1.0−45 Formula (I)

2. The inorganic nanoparticle labeling agent, as described in the foregoing 1, wherein the inorganic nanoparticles are semiconductor nanoparticles.

3. The inorganic nanoparticle labeling agent, as described in the foregoing 2, wherein the semiconductor nanoparticles each contain silicon (Si).

4. The inorganic nanoparticle labeling agent, as described in the foregoing 2 or 3, wherein the semiconductor nanoparticles have a core/shell structure and the composition of the core is different from that of the shell.

### Effect of the Invention

There can be provided an inorganic nanoparticle labeling agent exhibiting adaptability of being usable as a labeled material in the field of biology and medical science, and achieving fluorescence at a stable emission intensity.

That is to say, the surfaces of inorganic nanoparticles are modified with a specific organic compound under the specified embodying conditions, rendering it feasible to control hydrophilicity and non-specific bonding property within a living tissue and to be suitably usable as a labeled material to know the dynamic state of biomolecules.

### PREFERRED EMBODIMENTS OF THE INVENTION

The inorganic nanoparticle labeling agent of the invention is featured in that the inorganic nanoparticle labeling agent comprises inorganic nanoparticles, each having a surface modified with an organic compound, wherein the inorganic nanoparticles exhibit an average particle size of 1 to 10 nm, the organic compound is a compound containing a polyethylene glycol chain, the average particle size D of the inorganic nanoparticle labeling agent is from 8 to 25 nm, and the amount M (mol) of the organic compound per inorganic nanoparticle and the length L (nm) of the organic compound, measured on the inorganic nanoparticle surface are related by the foregoing formula (I). This feature is a technical feature in common with the invention set forth in the foregoing 1 to 4.

In the embodiments of the invention, the inorganic nanoparticles preferably are semiconductor nanoparticles. The semiconductor nanoparticles preferably comprise at least one of silicon (Si) and germanium (Ge). Further, the semiconductor nanoparticles preferably have a core/shell structure, in which the composition of the core is different from that of the shell.

In the following, there will be described constituent elements of the invention and preferred embodiments of the invention.

### Inorganic Nanoparticle

Materials of inorganic nanoparticles related to the invention may use various fluorescence-emitting compounds known in the art and their raw materials. In addition to semiconductor materials described later, there can be used, for example, rare earth elements such as erbium (Er), holmium (Ho), praseodymium (Pr), thulium (Tm), neodymium (Nd), gadolinium (Gd), europium (Eu), ytterbium (Yb), samarium (Sm) and cerium (Ce) and halogen compounds containing these elements.

In the invention, it is preferred to use, as inorganic nanoparticles, semiconductor nanoparticles described below.

### Semiconductor Nanoparticle

Materials used for the semiconductor nanoparticles related to the invention may employ various fluorescence-emitting compounds known in the art and raw materials for them. For instance, various semiconductor material which have been known as a material used for semiconductor nanoparticles may be employed as a raw material. Specifically there may be employed, for example, semiconductor compounds of group IV, group II-VI, and group III-V of the periodic table and raw material compounds containing elements constituting the semiconductor materials.

Examples of a group II-VI semiconductor include MgS, MgSe, MgTe, CaS, CaSe, CaTe, SrS, SrSe, SrTe, BaS, BaSe, BaTe, ZnS, ZnSe, ZnTe, CdS, CdSe, HgS, HgSe and HgTe.

Examples of a group III-V semiconductor include GaAs, GaN, GaP, GaSb, InGaAs, InP, InN, InSb, InAs, AlAs, AlP, AlSb and AlS.

Among group IV semiconductors, Ge and Si are specifically suitable.

Among the foregoing semiconductor materials, Si, Ge, ZnS, InN and InP are specifically preferred in terms of composition meeting safety and further of these, silicone (Si), zinc (Zn) and germanium (Ge) are specifically preferred as a main component atom forming the semiconductor nanoparticles of the invention. In the invention, the expression, “the main component atom forming the semiconductor nanoparticles” refers to an atom exhibiting the maximum content among atoms forming the semiconductor nanoparticles.

In the present invention, preferably, semiconductor phosphor nanoparticles have a core/shell structure. In such a case, it is preferred that semiconductor phosphor nanoparticles are those which have a core/shell structure constituted of a core particle of a semiconductor particle and a shell layer covering the core particle, and that the core particle differs in chemical composition from the shell layer. Accordingly, it is preferred that the band gap of the shell is higher than that of the core.

A shell is necessary to stabilize surface defects and enhance luminance and is also important to form the surface onto which a surface-modifying agent easily adsorbs. It is also an important constitution to achieve enhanced precision of the detection sensitivity for the effect of the invention.

There will be described a core particle and a shell layer.

### Core Particle

Semiconductor materials used for core particles may employ a various kinds of semiconductor materials. Specific examples thereof include MgS, MgSe, MgTe, CaS, CaSe, CaTe, SrS, SrSe, SrTe, BaS, BaTe, ZnS, ZnSe, ZnTe, CdS, CdSe, CdTe, GaAs, GaP, GaSb, InGaAs, InP, InN, InSb, InAs, AlAs, AlP, AlSb, AlS, PbS, PbSe, Ge, Si and a mixture of these. In the invention, a specifically preferred semiconductor material is Si, Zn or Ge.

The average particle size of the core related to the invention is preferably from 0.5 to 15 nm.

In the invention, the average particle size of semiconductor phosphor nanoparticles needs to be determined three-dimensionally but it is difficult to determine the particle size in such a manner because of its being extremely minute. Actually, it has to be determined in a two-dimensional image, so that it is preferred to determine an average size in such a manner that electron micrographs are taken using a transmission electron microscope (TEM) to perform averaging. Thus, electron micrographs are taken using a TEM and a sufficient number of particles are measured with respect to cross-sectional area to determine the diameter of a circle, equivalent to the cross-sectional area and an arithmetic average thereof is defined as the average particle size. The number of particles to be photographed by a TEM is preferably at least 100 particles.

In the semiconductor nanoparticles related to the invention, the average core particle size is preferably controlled so that the nanoparticles emit fluorescence at the wavelength in the infrared region, that is, infrared-emit.

### Shell Layer

Semiconductor materials used for a shell may employ various kinds of semiconductor materials. Specific examples thereof include ZnO, ZnS, ZnSe, ZnTe, CdO, CdS, CdSe, CsTe, MgS, MgSe, GaS, GaN, GaP, GaAs, GaSb, InAs, InN, InP, InSb, AlAs, AlN, AlP, AlSb and further mixtures of these.

In the invention, the specifically preferred semiconductor material is SiO₂, GeO₂ or ZnS.

The shell layer related to the invention need not completely cover all of the surface of a core particle unless partial exposure of the core particle has an adverse effect

### Dopant

The semiconductor nanoparticles of the invention contain a heteroatom or an atomic pair of the heteroatom, as a dopant, and such a heteroatom is identical in valence electron configuration with a main component atom forming the semiconductor nanoparticles and the dopant is uniformly distributed on or near the surface of the semiconductor nanoparticles.

Herein, “valence electron” refers to an electron which belongs to the outermost shell of electron shells (K shell, L shell, M shell, etc) constituting an atom. Therefore, in cases when the main component atom forming the semiconductor nanoparticles is silicon (Si), the valence electron is of four electrons in the outermost shell and an atom or an atomic pair having an equivalent valence electron configuration includes, for example, Be—Be (a Be pair), Mg—Mg (a Mg pair) and Ge.

In cases when the component atom forming the semiconductor nanoparticles of the invention is silicon (Si) or germanium (Ge), the dopant is preferably Be—Be.

In the invention, the location in which a dopant is contained is required to be on the surface of the semiconductor nanoparticles or near the surface of the semiconductor nanoparticles. Herein, “near the surface” is the region from the surface of the semiconductor nanoparticles to 30% of the radius of the nanoparticles, and preferably 15%.

The distribution of a dopant can be observed or measured by X-ray photoelectron spectrometry (XPS/ESCA; XPS: X-ray Photoelectron Spectroscopy/ESCA: Electron Spectroscopy for Chemical Analysis). The X-ray photoelectron spectrometry is a method to investigate the state (for example, element composition) of the solid surface or in the vicinity thereof by measuring the kinetic energy of an electron ejected upon exposure to monochromatic light (X-ray).

### Particle Size of Semiconductor Nanoparticle

The average particles size of the semiconductor nanoparticles related to the invention is preferably from 1 to 10 nm and more preferably from 1 to 5 nm.

It is well-known that, of semiconductor nanoparticles related to the invention, in nano-sized particles having a smaller particle size than the electron wavelength (approximately, 10 nm), in which the influence of finiteness of size on the motion of electrons, as the quantum size effect becomes larger, exhibit a specific physical property differing from the bulk body. In general, semiconductor nanoparticles which are a nanometer-sized semiconductor substance and exhibit a quantum confinement effect are also called “quantum dot”. Such a quantum dot is a minute mass within ten and some nm, collected of some hundreds to some thousands semiconductor atoms and liberates an energy corresponding to the energy band gap of the quantum dot when it reaches an energy-excited state on absorption of light from an excitation source. Accordingly, control of the energy band gap can be achieved by controlling the size or material composition of a quantum dot, whereby energy of wavelength bands at various levels can be employed. Further, a quantum dot, that is, semiconductor nanoparticles are featured in that the emission wavelength can be controlled by variation of particle size on the same composition.

Semiconductor nanoparticles related to the invention can be controlled so as to exhibit fluorescence in the range of 350 to 1100 nm but in the invention, to minimize effects of the emission of a living body cell and achieve enhanced SN ratio, an emission of a wavelength in a near-infrared region is preferably used.

### Production Method of Semiconductor Nanoparticles

Semiconductor nanoparticles related to the invention can be produced by a liquid phase process or gas phase process known in the art.

Production methods by a liquid phase process include, for example, a coprecipitation method, a sol-gel method, a homogeneous precipitation method and a reduction method. There are further included methods superior in production of nanoparticles, such as a reverse micelle method and a supercritical hydrothermal synthesis method (as described in, for example, JP 2002-322468A, JP 2005-239775A, JP 10-310770A, and JP 2000-104058A).

A producing method of an assembly of semiconductor phosphor nanoparticles is preferably a method comprising a step of reducing a semiconductor material precursor through reduction reaction. Further, in one preferred embodiment of the invention, the reaction of such a semiconductor material precursor is performed in the presence of a surfactant. A semiconductor material precursor related to the invention is a compound containing an element used for the above-described semiconductor material and, for example, in the case of the semiconductor material being Si, SiCl₄ is cited as a semiconductor material precursor. Other examples of a semiconductor material include InCl₃, P(SiMe₃)₃, ZnMe₂, CdMe₂, GeCl₄ and tributylphosphine selenium.

The reaction temperature is not specifically limited if it is not less than the boiling point of the semiconductor material precursor and not more than the boiling point of the solvent, but is preferably in the range of 70 to 110° C.

### Reducing Agent

A reducing agent used for reduction of a semiconductor material precursor can be chosen from a variety of reducing agents known in the art, in accordance with reaction conditions. In the invention, reducing agents such as lithium aluminum hydride (LiAlH₄), sodium borohydride (NaBH₄), sodium aluminum bis(2-methoxyethoxy)hydride, lithium tri(sec-butyl)borohydride [LiBH(sec-C₄H₉)₃], potassium tri(sec-butyl)borohydride and lithium triethylborohydride are preferred in terms of reducing strength. Of these, lithium aluminum hydride (LiAlH₄) is specifically preferred in terms of reducing strength.

### Solvent

A variety of solvents known in the art are usable as a solvent to disperse a semiconductor material precursor. Preferred examples thereof include alcohols such as ethyl alcohol, sec-butyl alcohol and t-butyl alcohol; and hydrocarbon solvents such as toluene, decane and hexane. A hydrophobic solvent such as toluene is specifically preferred as a solvent for use in these dispersion.

### Surfactant

There are usable a variety of surfactants known in the art in the invention, including anionic, non-ionic, cationic, and amphoteric surfactants. Of these are preferred quaternary ammonium salts, such as tetrabutylammonium chloride, bromide, or hexafluorophosphate; tetraoctylammonium bromide (TOAB), and tributylhexadecylphosphonium bromide.

A reaction by a liquid phase process is greatly variable according to the state of a compound in liquid including a solvent. There is required attention specifically when producing nano-sized particles superior in mono-dispersibility. In a reverse micelle method, for example, the size or state of reversed micelles which forms a reaction field is varied by the concentration or kind of a surfactant used therein, so that the condition to form nanoparticles is restricted. Accordingly, an appropriate surfactant is required to be combined with a solvent.

Production methods by a gas phase process include (1) a method in which a raw material semiconductor is evaporated by a first high temperature plasma generated between opposed electrodes and allowed to pass through a second high temperature plasma generated through electrodeless discharge in a reduced pressure environment (as described in, for example, JP 6-279015A), (2) a method in which nanoparticles are separated from an anode composed of a raw semiconductor material through electrochemical etching (described in, for example, JP 2003-515459A, (3) a laser ablation method (described in, for example, JP 2004-356163A), and (4) a high-speed sputtering method (described in, for example, JP 2004-296781A). There is also preferably employed a method in which a raw material gas is subjected to a gas phase reaction in a low pressure state to synthesize a powder containing particles.

### Post-Treatment After Formation of Semiconductor Nanoparticle

In the production method of semiconductor nanoparticles, it is preferred that any one of post-treatment by plasma, heating, radiation or ultrasonic waves is included after formation of semiconductor nanoparticles, specifically after shell formation.

An appropriate plasma treatment may be chosen from low temperature/high temperature plasma, microwave plasma and atmospheric plasma, of which the microwave plasma is preferred.

A heat treatment can be chosen among atmosphere, vacuum and inert gas regions and applied heating, and the applied temperature range differs, depending on the constitution of phosphor particles. An excessively high temperature often causes strain or flaking between the core and the shell. A low temperature results in poor effect and a range of 100 to 300° C. is preferably employed.

A radiation treatment employs high-energy X-rays, γ-rays or neutron rays, or low-energy vacuum ultraviolet (UV) rays, ultraviolet rays or short-pulse laser rays. Treatment time depends on the kind of a radiation. For instance, X-rays, which exhibit high penetrability, often perform exposure within a relatively short time; on the contrary, ultraviolet rays require exposure over a relatively longtime.

Effects of these post-treatments are not elucidated in principle but it is assumed that adhesiveness at the interface between core and shell is reinforced and passivation is accelerated, resulting in enhanced emission efficiency. It is also assumed that such an influence is remarkable in an infrared emitter and is reflected in its characteristics.

In the invention, the band gap of a shell is preferably higher than that of its core. A shell is needed to stabilise surface defects on the core particle surface and to achieve enhanced luminance, and is also important to form a surface onto which a surface-modifying agent is easily adhered, when used as a fluorescent labeling agent.

### Surface Modification of Inorganic Nanoparticle

The inorganic nanoparticle labeling agent of the invention is one containing inorganic nanoparticles which were surface-modified with an organic compound, wherein the inorganic nanoparticles exhibit an average particle size of from 1 to 10 nm, the organic compound is a compound having a poly(ethylene glycol) chain, the average particle size D of the inorganic nanoparticle labeling agent is from 8 to 25 nm, and the amount M (expressed in mol) of the organic compound per a single particle of the inorganic particles and the length L (expressed in mm) of the organic compound which is measured from the inorganic particle surface, meet the relationship represented by the following formula (I):

(M×10²²)×L/D=1.0−4.5 Formula (I)

The method of surface modification may employ various methods known in the art. Basically in the invention, for example, the surfaces of inorganic particles are hydroxylated with an aqueous hydrogen peroxide. Subsequently, the thus hydroxylated surface is allowed to react with a silane coupling agent containing a functional group such as a mercapto group or/and an amino group. Then, a compound containing a polyethylene glycol chain (such as polyethylene glycols) which also contains a functional group capable of reacting with the functional group described above is allowed to react, whereby the surface-modified, inorganic nanoparticle labeling agent related to the invention is prepared.

In the invention, there are usable various compounds having a molecular weight of 300 to 3000 as the foregoing compound containing a polyethylene glycol chain (such as polyethylene glycols) and examples thereof include polyethylene glycols containing, at the end, an amino group, a carboxyl group or a maleimido group which targets a biomolecule.

There are usable a silane compound represented by the following formula or its derivatives as a silane coupling agent usable in the invention.

X-A-Si(OR)ₙR′ₙ₋₃ Formula:

wherein X is a functional group, such as an amino group, a mercapto group, a halogen atom, an epoxy group, a vinyl group, a methacryloyl group or an N-(aminoalkyl)-amino group.

Further, A is a hydrocarbon chain, including, for example, —(CH₂)₂—, —(CH₂)₃— and —(CH₂)₄—. In the foregoing formula, R and R′ may be the same or different and are each a straight-chain or branched alkyl group having 1-6 carbon atoms.

Representative examples of the silane coupling agent include N-(2-aminoethyl)-3-aminopropylmethyldimethoxysilane, N-(2-aminoethyl)-3-aminopropyltrimethoxysilane, N-(2-aminoethyl)-3-aminopropyltriethoxysilane, 3-aminopropyltrimethoxysilane, 3-aminopropylmethoxysilane, 3-triethoxysilyl-N-(1,3-dimethyl-butylidene)propylamine, N-phenyl-3-aminopropyltrimethoxysilane, N-(vinylbenzyl)-2-aminoethyl-3-aminopropyltromethoxysilane, 3-ureidopropylethoxysilane, 3-chloropropyltrimethoxysilane, 3-mercaptopropylmethyldimethoxysilane, 3-mercaptopropyl-trimethoxysilane, bis(triethoxysilylpropyl)tetrasulfide, 3-isocyanatopropyltriethoxysilane, 2-(3,4-epoxycyclohexyl)-ethyltrimethoxysilane, 3-glycidoxypropylmethyldiethoxysilane, 3-glycidoxypropyltriethoxysilane, 3-methacryloxypropylmethyl-diethoxysilane, 3-methacryloxypropyltriethoxysilane, 3-acrylyloxypropyltrimethoxysilane, p-styryltrimethoxysilane, vinyltrichlorosilane, vinyltrimethoxysilane, vinyltriethoxysilane, vinyltriacetoxysilane, and octadecyldimethyl[3-(trimethoxysilyl)propyl]ammonium chloride.

Of the foregoing compounds, a silane compound containing an amino or mercapto group is preferably used in the invention.

A coupling agent is usable in the form of a solution diluted with a solvent described below, which is generally used as an aqueous solution but may be used in the form of an aqueous solution added with a small amount of acetic acid. A coupling agent is used at an appropriately concentration and, for example, a solution with a concentration of 0.001 to 5.0% or 0.01 to 1.0% may be added to a dispersion of inorganic nanoparticles.

A dispersing solvent usable in the invention is not necessarily fixed, depending on solubility of the surface-modifying compound, but examples of such a solvent include water; ketones such as acetone and methyl ethyl ketone; esters such as ethyl acetate; alcohols such as methanol and ethanol; non-protonic polar solvent such as dimethyl formamide, dimethyl sulfoxide, sulfolane, diglyme and hexamethylphosphoric triamide; and other compounds such as nitromethane and acetonitrile. Specifically, water and hydrophilic solvents such as an aqueous alcohol or ketone are suitably usable.

The present invention is featured in that the organic compound to modify the inorganic nanoparticle surface is a compound containing a polyethylene glycol chain, the inorganic nanoparticle labeling agent exhibits an average particle size D of from 8 to 25 nm, and the amount M (expressed in mol) of the organic compound per a single particle of the inorganic particles and the length L (expressed in mm) of the organic compound from the inorganic particle surface meet the relationship represented by the foregoing formula (I).

Therefore, to control so that the average particle size of an inorganic nanoparticle labeling agent falls within the range of from 8 to 25 nm and the amount and length of the organic compound fall within the range of the relationship (I), it is required to choose the kind of polyethylene glycols and silane coupling agents and to control the used amount thereof.

The average particle size of an inorganic nanoparticle labeling agent can be determined by electron-microscopic observation in accordance with the method to determine the average particle size of semiconductor nanoparticles, as described earlier.

The amount of the organic compound (polyethylene glycols) can be determined through spectroscopic measurement of the compound remaining in the reaction mixture after completion of reaction by using an ultraviolet-visible absorption spectrometer, or in such a manner that, after completing surface modification, the organic compound is completely separated from inorganic nanoparticles through acid decomposition and its amount is spectroscopically determined similarly to the foregoing.

The length L of an organic compound of the inorganic nanoparticle labeling agent, which is a length measured from the inorganic nanoparticle surface, is determined in such a manner that the inorganic nanoparticle size before being modified and the labeling agent particle size after being surface-modified are each measured by using a particle size measurement apparatus employing a laser dynamic light scattering method or TEM and the difference between them is calculated. Namely, the length L is determined by calculating this difference.

With respect to the amount M of an organic compound per inorganic nanoparticle, the amount of the organic compound on the particle surfaces which is determined from reduction of mass by using a TGA (thermogravimetric analyzer) and the number of nanoparticles is determined from the amount of inorganic particles and particle size, from which the amount M is determined.

### Inorganic Nanoparticle Labeling Agent

The semiconductor nanoparticles of the invention, of which the surface is provided with an appropriate surface-modifying agent, is applicable to a fluorescent labeling agent to fluorescence-label a targeted substance (or a target). Specifically, a surface-modifying compound which is affinitive to or connective to a living body is disposed on the particle surface, which is suitably used as a biomolecule fluorescence labeling agent (biosubstance fluorescence labeling agent) to fluorescence-label a targeted substance such as a protein or a peptide.

When used as a biomolecule fluorescence labeling agent (biosubstance fluorescence labeling agent), it is preferred in terms of non-invasiveness and penetrability for living tissue to control an emission characteristic through particle size, or the like so that infrared light is emitted by excitation of near-infrared to infrared.

In the invention, a surface-modifying compound preferably is one which contains at least one functional group and at least one group capable of bonding to a semiconductor nanoparticle. The latter is a hydrophobic group capable of adsorbing to a hydrophobic semiconductor nanoparticle and the former is a functional group which is affinitive with a living substance and capable of bonding a biomolecule. Surface-modifying compounds may use a linker which allows them to be combined with each other.

A group capable of bonding to a semiconductor nanoparticle may be any functional group capable of bonding to a semiconductor material to form semiconductor nanoparticles. In the invention, such a functional group preferably is a mercapto group (or a thiol group).

Examples of a functional group capable of affinity-bonding to a biosubstance include a carboxy group, an amino group, a phosphonic acid group and a sulfonic acid group.

Herein, the biosubstance refers to a cell, DNA, RNA, oligonucleotide, protein, antigen, antibody, endoplasmic reticulum, nuclear, a Golgi body and the like.

To be allowed to bond to semiconductor nanoparticles, a mercapto group may be allowed to bond by adjusting the pH to a value suitable for surface modification. To the other end is introduced an aldehyde group, an amino group or a carboxyl group to form a peptide bonding with an amino group or a carboxyl group. Introduction of an amino group, an aldehyde group or a carboxyl group to DNA, oligonucleotide or the like can similarly form a bond.

Specific examples of a method of preparing a biomolecule fluorescent labeling agent (biosubstance fluorescent labeling agent) include a method in which hydrophilized semiconductor nanoparticles are linked to a molecule labeling substance via an organic molecule. In a biomolecule fluorescent labeling agent (biosubstance fluorescent labeling agent) prepared by this method, a molecular labeling substance specifically bonds to and/or reacts with a targeted a biosubstance, making it feasible to perform fluorescence labeling of the biosubstance.

Examples of the molecule labeling substance include a nucleotide chain, antigen, antibody, and cyclodextrin.

Any organic molecule, which is capable of linking a semiconductor nanoparticle and a molecular labeling agent, is not specifically limited and, for example, among proteins, albumin, myoglobin or casein, or biotin together with avidin is preferable. The binding mode is not specifically limited, including a covalent bond, ionic bond, hydrogen bond, coordination bond, physical adsorption and chemical adsorption. Of these, a bonding with high bonding strength, such as a covalent bond is preferred in terms of bonding stability.

Specifically, in the case of semiconductor phosphor nanoparticles being hydrophilized with mercaptoundecanoic acid, avidin is used together with biotin. In that case, carboxyl groups of the hydrophilized nanoparticles are appropriately covalent-bonded to avidin, further, this avidin is selectively bonded to biotin and this biotin is bonded to a biomaterial labeling agent to form a biomaterial labeling agent.

### Hydrophilization of Semiconductor Nanoparticle

The particle surface of the foregoing semiconductor nanoparticle assembly is generally hydrophobic. For example, in cases when used as a biomaterial labeling agent, the particles are poorly dispersed in water as they are, producing problems such as coagulation. Accordingly, it is preferred to subject the surface of semiconductor phosphor nanoparticles to a hydrophobilization treatment.

Such a hydrophobilization treatment is conducted, for example, in such a manner that after removal of hydrophobic substances with pyridine or the like, a surface-modifier is chemically or physically bound to the particle surface. A preferred surface-modifier is one containing a carboxyl or amino group as a hydrophilic group. Specific examples of such a surface-modifier include mercaptopropionic acid, mercaptoundecanoic acid and aminopropane-thiol. Specifically, for example, 10⁻⁵ g of core/shell type Ge/GeO₂ nanoparticles are dispersed in 10 ml pure of water containing 0.2 g of mercaptoundecanoic acid and stirred at 40° C. for 10 min. to subject the shell surface to the treatment, whereby the shell surfaces of the nanoparticles are modified with a carboxyl group.

Specific preparation for surface modification of semiconductor nanoparticles may be conducted in accordance with methods, as described in, for example, Dabbousi et al., J. Phys. Chem. B101 (1997); Hines et al., J. Phys. Chem. 100: 468-471 (1996); Peng et al., J. Am. Chem. Soc. 119, 7019-7029 (1997); and Kuno et al., J. Phys. Chem. 106: 9869 (1997).

### Inorganic Nanoparticle Labeling Agent and Biomolecule Detection System by Use Thereof

The fluorescent labeling substance related to the invention, having the foregoing characteristic, is suitably applicable to a biomolecule detection system, feature in that the inorganic nanoparticle labeling agent is supplied to a living cell or a living tissue and fluorescence emitted by exciting semiconductor nanoparticles with radiation is detected, whereby a biomolecule in the targeted living cell or a living tissue is detected.

To a living cell or living body having a targeted (or traced) biomolecule is added an inorganic nanoparticle labeling agent according to the invention and is bound or adsorbed onto the targeted material; such a bound or adsorbed material is exposed to an exciting light of a prescribed wavelength and a fluorescence at a specific wavelength, which is emitted from semiconductor phosphor particles, is detected to perform fluorescent dynamic imaging of the targeted (or traced) material. Thus, a fluorescent labeling substance related to the invention can be employed for a bio-imaging method (technical means to visualize a bio-molecule constituting a biomaterial or its dynamic phenomenon).

Examples of radiation used for excitation include visible light of a halogen lamp or a tungsten lamp, an LED, a near-infrared laser light, an infrared laser light, X-rays, and γ-rays.

### Molecular and Cellular Imaging Method

The semiconductor nanoparticles of the invention is usable as a fluorescent labeling substance by allowing a probe molecule (molecule for searching) to be bound to a molecule existing in the interior or on the surface of cell tissue as a target.

In this application, “target” refers to a biomolecule targeted by semiconductor nanoparticles, which is, for example, a protein expressed preferentially in a tissue or a cell or a Golgi body, nucleus or membrane protein. Examples of an appropriate targeted material include enzymes, proteins, cell surface acceptors, nuclear acids, lipids and phospholipids, but are not limited to these.

In the invention, it is preferred to adopt an appropriate probe molecule corresponding to a targeted (measured) substance with the purpose of imaging of the interior of a living body, dynamic measurement of a substance within a cell or the like.

An inorganic nanoparticle labeling agent (biomolecule fluorescent labeling agent) employing semiconductor nanoparticles of the invention is applicable to various molecule-cell imaging methods known in the art. Examples thereof include molecule-cell imaging methods by a laser injection method, a microinjection method, an electroporation method or the like. Of these methods is preferred application to a molecule-cell imaging method by the laser injection method.

“Laser injection method” refers to an optical method in which a laser light is irradiated directly onto a cell to bore a minute hole to introduce an external substance such as a gene therethrough.

The microinjection method refers to a method in which an external substance such as a gene therethrough is mechanically introduced by air pressure using a minute needle (micropipette, microsyringe, or the like).

“Electroporation method” refers to a method in which electrical stimulation is applied to a cell to induce deformation of the cell to introduce an external substance such as a gene. For instance, employing an extracellular solution being introduced through a small pore formed in the cell membrane for a short period when a high voltage of some thousands V/cm is applies to a cell suspension at a pulse of some tens of microseconds, then a sample which is intended to be introduced, such as DNA is added to the extracellular solution and introduced into the cell.

### EXAMPLES

### Preparation of Inorganic Nanoparticle

Preparation of Si core particle and Si/SiO₂ core/shell particle

### HF Etching Method:

In preparation of inorganic phosphor nanoparticles (hereinafter, also denoted as “Si semiconductor particles” or “Si core particles”) through solution of thermally treated SiOₓ (X=1.999) in hydrofluoric acid, first, SiOₓ (X=1.999) film which was formed on a silicon wafer by plasma CVD was annealed in an inert gas atmosphere at 1100° C. Thereby, fine Si semiconductor particles were precipitated onto the SiO₂ film. Controlling an annealing time deposited fine Si particles differing in size.

Subsequently, this silicon wafer was treated with an aqueous 1% hydrofluoric acid solution at room temperature to remove the SiO₂ membrane, and there were obtained silicon (Si) semiconductor nanoparticles having a size of several nanometers which were aggregated on the liquid surface. A daggling bond (unpaired bond) of the silicon (Si) atom on the semiconductor particle (crystal) surface is hydrogen-terminated by the foregoing hydrofluoric acid treatment, whereby the silicon (Si) crystal is effectively stabilized. Thereafter, the surfaces of the thus obtained Si semiconductor particles were subjected to thermal oxidation by heating in an oxygen atmosphere at 800 to 1000° C. over approximately 1.5 hours to form a shell layer comprised of SiO₂ over a core comprised of a silicon semiconductor particle. The average particle size of inorganic phosphor nanoparticles composed of Si/SiO₂.core/shell was measured by using ZETA SIZER, produced by Sysmex Co., Ltd. and the result thereof is shown in Table 1.

### Preparation of Si/Zns Core/Shell Particle:

The thus obtained Si core particles were dispersed in pyridine and maintained at 100° C. Separately, Zn(C₂H₅)₂, [(CH₃)₃Si]₂S and P(C₄H₉)₃ were gradually mixed at 100° C. under an argon atmosphere, while applying ultrasonic waves.

The thus obtained mixture was dropwise added to pyridine dispersion. After completing addition, the mixture was controlled to 100° C. and slowly stirred for 30 minutes, while maintaining a pH of 8.0. The mixture was subjected to centrifugal separation and sedimented particles were collected. As a result of element analysis of the obtained particles, Si and ZnS were confirmed and it was proved from XPS analysis that the Si surface was covered with ZnS. The average particle size of inorganic phosphor nanoparticles was measured by using ZETA SIZER, produced by Sysmex Co., Ltd. and the result thereof is shown in Table 1.

### Introduction of Surface-Modifying Compound:

When labeling a living substance with the foregoing inorganic nanoparticles, it is necessary to introduce, to both the particles and the living substance, a functional group capable of bonding to both of them, which was conducted as follows.

### Introduction of Modification Group to Si/SiO₂ Core/Shell Particle:

Employing bonding between mercapto groups (SH groups), a carboxyl group is introduced to phosphor semiconductor particles.

First, the foregoing Si/SiO₂ core/shell particles are dispersed in an aqueous 30% hydrogen peroxide solution over 10 minutes to hydroxylate the crystal surface. Then, the solvent was replaced by toluene and thereto, mercaptopropyltriethoxysilane was added in an amount of 2% of toluene and stirred over two hours, whereby SiO₂ on the uppermost surface of the Si core particle was transformed to silane, at the end of which a mercapto group was simultaneously introduced. Subsequently, the solvent was replaced by water and a buffer salt was added thereto. Further, compounds (of a methoxy-terminal end) having introduced a mercapto group to one end and differing in polyethylene glycol chain length fitting to the compound length L, defined in the formula I, were chosen, as shown in Table 1 and each of the compounds was added in an excess amount and stirred for 3 hours. Reaction was performed with varying reaction conditions (reaction time, temperature, pH, and the presence/absence of a catalyst), whereby the surface coverage (M) was achieved, as shown in Table 1. Objective labeling agents were thus obtained. Using plural columns capable of absorbing the respective raw material components used for preparation of the obtained labeling agents and a final column provided with size selectivity by varying the bore diameter of the final column, a HPLC treatment was performed continuously or separately through all the columns to remove components such as raw materials, solvents and the like, except a label A.

Further, fine control of the size of labeling agents shown in Table 1 was performed by employing the length of a polyethylene glycol chain.

### Introduction of Modification Group to Si/ZnS Core/Shell Particle:

Si/ZnS core/shell particles obtained above were dispersed in a buffer salt solution. Further thereto, a PEG compound (with a methoxy group at one end) having introduced a mercapto group at the other end and similar to the foregoing compound was added in an excess amount and stirred for two hours to obtain a labeling agent having a mercapto group bonded to the particle surface. Using plural columns capable of absorbing the respective raw material components used for preparation of the obtained labeling agents and a final column provided with size selectivity by varying the bore diameter of the final column, a HPLC treatment was performed continuously or separately through all the columns to remove components such as raw materials, solvents and the like, except for a label A. The labeling agent size was controlled by controlling the PEG length.

Thereby, a surface-modifying compound having a length of L shown in Table 1 was introduced to the surface. Reaction was performed with varying reaction conditions (reaction time, temperature, pH, and the presence/absence of a catalyst), whereby a surface-modifying compound, in an amount M, was introduced onto the particle surface, as shown in Table 1.

The length (L) of a surface-modifying compound, measured from the particle surface was determined in such a manner that the particle size was measured in advance and the particle size after being surface-modified was measured again, and from the difference thereof, the length (L) was determined. The particle size can be determined by using a measuring apparatus employing a laser dynamic light scattering method or a TEM (transmission electron microscope).

With respect to the amount of a surface-modifying compound per particle, the number of particles was determined by dividing the mass of used particles by a product of a volume calculated from a particle size and a specific gravity. After completing surface modification, surface-modified particles were subjected to TGA (thermogravimetric analysis) to determine mass reduction due to burning a surface-modifying compound, from which the surface-modifying compound amount was determined. This amount was divided by the number of particles to determine the average surface modification amount per particle.

### Fluorescence-Labeled Biomolecule Observation Example:

The above-obtained labeling agent was mixed with an equivalent concentration of a sheep serum albumin (SSA) to be individually incorporated into Vero cells. After performing culture at 37° C. for two hours, the thus obtained labeling materials were subjected to a trypsinization treatment, dispersed in a 5% FBS-containing DMEM culture medium and then sowed into a glass bottom dish. The cells cultured at 37° C. overnight were solidified with 4% formalin and nuclei were dyed with DAPI, and then, fluorescence observation was performed by using a confocal laser-scanning microscope (excitation wavelength: 405 nm).

The state of a labeling agent being introduced to cellular endosomes and accumulated in a membrane protein was evaluated with respect to density depending on emission intensity and dispersion state. Namely, a labeling agent which was introduced into a cell and efficiently and uniformly moved to and accumulated in endosomes, resulted in an enhanced emission intensity and uniform distribution with a broad area. This reflected the state of the labeling agent without coagulation or bonding. On the contrary, a labeling agent which was introduced under influences of coagulation and non-specific adsorption and moved inefficiently, resulted in fluorescence with reduced intensity and in non-uniform patches, in which emission intensity varied depending on location and the accumulated emission area was so small. The result of such observation is shown in Table 1.

TABLE 1

| | | | | Modification | | | | |
|---|---|---|---|---|---|---|---|---|
| | | | | Amount of | | Stability: | | |
| | Nano- | | Labeling | Organic | | Variation | | |
| | particle | Nano- | Agent | Compound: | | in | | |
| | Size | particle | Size: D | M × 10²² | (M × 10²²) × | Emission | | |
| No. | (nm) | Species | (nm) | (mol/particle) | L/D | Intensity | Cell Imaging Observation | Remark |
| 1 | 4.0 | Si/SiO₂ | 14 | 2.8 | 1.0 | 13% | Emission being uniform, and shape of cell | Inv. |
| | | | | | | | membrane being clearly observed | |
| 2 | 4.0 | Si/SiO₂ | 9 | 6.5 | 1.8 | 8% | Emission being uniform, and shape of cell | Inv. |
| | | | | | | | membrane being clearly observed | |
| 3 | 9.0 | Si/SiO₂ | 19 | 10.5 | 2.8 | 15% | Emission being uniform, and shape of cell | Inv. |
| | | | | | | | membrane being clearly observed | |
| 4 | 9.0 | Si/SiO₂ | 14 | 24 | 4.3 | 13% | Emission being uniform, and shape of cell | Inv. |
| | | | | | | | membrane being clearly observed | |
| 5 | 4.0 | Si/SiO₂ | 9 | 2 | 0.6 | 33% | Coagulation markedly occurring, emission | Comp. |
| | | | | | | | being nonuniform, and membrane being | |
| | | | | | | | invisible in some portions | |
| 6 | 9.0 | Si/SiO₂ | 19 | 18 | 4.7 | 35% | Coagulation occurring, emission being | Comp. |
| | | | | | | | nonuniform, and membrane being unclear | |
| | | | | | | | and greatly varied depending on area | |
| 7 | 4.0 | Si/ZnS | 14 | 2.8 | 1.0 | 10% | Emission being uniform, and shape of cell | Inv. |
| | | | | | | | membrane being clearly observed | |
| 8 | 4.0 | Si/ZnS | 9 | 6.5 | 1.8 | 6% | Emission being uniform, and shape of cell | Inv. |
| | | | | | | | membrane being clearly observed | |
| 9 | 9.0 | Si/ZnS | 19 | 10.5 | 2.8 | 12% | Emission being uniform, and shape of cell | Inv. |
| | | | | | | | membrane being clearly observed | |
| 10 | 4.0 | Si/ZnS | 14 | 16 | 5.7 | 34% | Coagulation occurring, emission being | Comp. |
| | | | | | | | nonuniform, and membrane being unclear | |

As shown in Table 1, it was proved that phosphor labeling compounds related to the present invention caused no coagulation, were superior in dispersibility, and being stable and clear in detectability for labeled living material.

1. An inorganic nanoparticle labeling agent comprising inorganic nanoparticles surface-modified with an organic compound, wherein the inorganic nanoparticles exhibit an average particle size of 1 to 10 nm, the organic compound is a compound containing a polyethylene glycol chain, the average particle size D of the inorganic nanoparticle labeling agent is from 8 to 25 nm; and an amount M (mol) of the organic compound per inorganic nanoparticle and a length L (nm) of the organic compound measured from an inorganic nanoparticle surface meet the relationship represented by the following formula (I):
(M×10²²)×L/D=1.0−4.5 Formula (I)

2. The inorganic nanoparticle labeling agent, as claimed in claim 1, wherein the inorganic nanoparticles are semiconductor nanoparticles.

3. The inorganic nanoparticle labeling agent, as claimed in claim 2, wherein the semiconductor nanoparticles contain silicon (Si).

4. The inorganic nanoparticle labeling agent, as claimed in claim 2, wherein the semiconductor nanoparticles have a core/shell structure and a composition of a core is different from that of a shell.

5. The inorganic nanoparticle labeling agent, as claimed in claim 3, wherein the semiconductor nanoparticles have a core/shell structure and a composition of a core is different from that of a shell.

6. A method of producing an inorganic nanoparticle labeling agent comprising surface-modified inorganic nanoparticles, the method comprising:
preparing inorganic nanoparticles, and
subjecting the inorganic nanoparticles to surface modification with an organic compound to form the surface-modified inorganic nanoparticles,
wherein the inorganic nanoparticles exhibit an average particle size of 1 to 10 nm, the organic compound is a compound containing a polyethylene glycol chain, the average particle size D of the inorganic nanoparticle labeling agent is from 8 to 25 nm; and an amount M (mol) of the organic compound per inorganic nanoparticle and a length L (nm) of the organic compound measured from an inorganic nanoparticle surface meet the relationship represented by the following formula (I):
(M×10²²)×L/D=1.0−4.5 Formula (I)

7. The method, as claimed in claim 6, wherein the inorganic nanoparticles are semiconductor nanoparticles.

8. The method, as claimed in claim 7, wherein the semiconductor nanoparticles contain silicon (Si).

9. The method, as claimed in claim 7, wherein the semiconductor nanoparticles have a core/shell structure and a composition of a core is different from that of a shell.

10. The method, as claimed in claim 8, wherein the semiconductor nanoparticles have a core/shell structure and a composition of a core is different from that of a shell.
